(19) United States
(12) Patent Application Publication
(10) Pub. No.: US 2016/0058475 A1
(43) Pub. Date: Mar. 3, 2016

(54) Polymer Osteosynthesis/Translaminar Screw for Surgical Spine Treatment

(71) Applicant: Jose Guilherme de Pinho Velho Wanderley, Teresopolis (BR)

(72) Inventor: Jose Guilherme de Pinho Velho Wanderley, Teresopolis (BR)

(21) Appl. No.: 14/853,295

(22) Filed: Sep. 14, 2015

Related U.S. Application Data
(63) Continuation of application No. PCT/IB2014/000379, filed on Mar. 17, 2014.
(60) Provisional application No. 61/787,179, filed on Mar. 15, 2013.

(51) Int. Cl. A61B 17/70; A61B 17/34; A61B 19/00; A61B 17/86; A61B 17/88

(52) U.S. Cl. CPC A61B 17/70; A61B 17/866; A61B 17/863; A61B 17/8875; A61B 17/7055; A61B 19/46; A61B 17/3472; A61B 10/0233

(57) ABSTRACT

A translaminar screw is formed from a polymer material (such as PEEK, PLLA, PCL, carbon fiber PEEK, and the like) so that the screw does not come loose, even after an extended period of mobilization. Spinal implants, instrumentation, and methods relating to stabilization and/or fusion of a facet joint via trans-facet and intra-facet delivery of the implants are disclosed herein. The implant or screw functions as a sort of flexible mechanical staple and/or key that prevents sliding motion between the diarthroidal surfaces of the facet joint. The spinal implant includes an elongated member extending from a distal tip to a proximal end having a head formed thereon. The elongated member can further include a threaded portion. The implant member can be, for example, a polymer translaminar screw that is formed from one of a PEEK, PLLA, PCL, carbon fiber PEEK, or similar polymer or other relatively flexible material.

### CROSS-REFERENCE TO RELATED APPLICATIONS

This application is a continuation of International Application No. PCT/IB2014/000379, filed Mar. 17, 2014, which claims priority from U.S. Provisional Application No. 61/787,179, filed Mar. 15, 2013. The disclosures of both applications are incorporated herein by reference.

### BACKGROUND OF THE INVENTION

This invention relates to the general field of orthopedic surgical implants. In particular, this invention relates to an osteosynthesis/translaminar screw that is formed from a polymer material (such as, for example, PEEK (polyether ether ketone), PLLA (poly-l-lactide acid), PCL (polycaprolactone), carbon fiber PEEK, and the like) and can be used in the field of surgical spine treatment and other applications.

The vertebrae in a patient's spinal column are linked to one another by intervertebral discs and facet joints. This three joint complex controls the movement of the vertebrae relative to one another. Each vertebra has a first pair of articulating surfaces located on the left side and a second pair of articulating surfaces located on the right side, and each pair includes a superior articular surface and an inferior articular surface. Together, the superior and inferior articular surfaces of the adjacent vertebrae form facet or zygapophyseal joints. Facet joints are synovial joints, which means that each joint is surrounded by a capsule of connective tissue and produces a fluid to nourish and lubricate the joint. The joint surfaces are coated with cartilage, allowing the joints to move or articulate relative to one another. Diseased, degenerated, impaired, or otherwise painful facet joints and/or discs can require surgery to restore function to the three joint complex. In the lumbar spine, for example, one form of treatment to stabilize the spine and to relieve pain involves fusion of the facet joint.

Pedicles connect the vertebral body to the posterior elements. Each vertebra has two pedicles. A basic pedicle screw structure includes a threaded shaft portion having one or more slots provided on a head portion. Pedicle screws are screwed into the spine through the respective pedicles, and a rod is used to lock the pedicle screws in place to minimize relative motion. These rods are locked into place with the pedicle screws using a fastening screw, such as a set screw.

Translaminar screw fixation on the lumbar spine, in context of spinal fusion and operative treatment of injuries, has been used for almost twenty five years. The principle of translaminar screw fixation consists of the use of osteosynthesis screws to lock the facet or zygapophyseal joints to prevent any possible movement between two vertebrae, with resulting immobilization of the two vertebrae. The screw enters on one side of the spinous process of the bone, extends through the mutual lamina, traverses the zygapophyseal joints (facet joints), and ends up in the base of transverse process of the lower vertebrae.

This method of spinal fusion with translaminar fixation has been known to fail because the implants have been made out of stainless steel and/or titanium alloy materials. Such metallic screws can become loose when more than six weeks of mobilization is stimulated. Thus, there is a need for an improved implant for use with translaminar fixation and other spinal and/or orthopedic procedures.

### SUMMARY OF THE INVENTION

In accordance with this invention, when the translaminar screw is formed from a polymer material (such as PEEK, PLLA, PCL, carbon fiber PEEK, and the like), the screw does not come loose, even after an extended period of mobilization. This could be attributed to the fact that PEEK and other polymer materials have elastic modulus properties that are similar to bone. Alternatively, it may be the result because PEEK and other polymer materials, due to their elasticity, can deform and again re-form to the position together with bone leading to flexible stabilization of the joint.

Spinal implants, instrumentation, and methods relating to stabilization and/or fusion of a facet joint via trans-facet and intra-facet delivery of the implants are disclosed herein. In general, the implant or screw functions as a sort of flexible mechanical staple and/or key that prevents sliding motion between the diarthroidal surfaces of the facet joint.

In the preferred embodiment, the spinal implant includes an elongated member extending from a distal tip to a proximal end having a head formed thereon. The elongated member can further include a threaded portion. The implant member can be, for example, a polymer translaminar screw that is formed from one of a PEEK, PLLA, PCL, carbon fiber PEEK, or similar polymer or other relatively flexible material.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a rear elevational view of a portion of a spine showing a conventional pedicle screw stabilization structure.

FIG. 2 is a side elevational view of the spine and the conventional pedicle screw stabilization structure illustrated in FIG. 1.

FIG. 3 is a rear elevational view of a portion of a spine showing a conventional translaminar screw fixation technique developed by Friedrich Magerl.

FIG. 4 is a side elevational view of the spine and the conventional translaminar screw fixation illustrated in FIG. 3.

FIG. 5 is a perspective view of a first embodiment of a translaminar screw in accordance with this invention.

FIG. 6 is a side elevational view of the first embodiment of the translaminar screw illustrated in FIG. 5.

FIG. 7 is a sectional elevational view of the first embodiment of the translaminar screw illustrated in FIGS. 5 and 6.

FIG. 8 is a perspective view of a second embodiment of a translaminar screw in accordance with this invention.

FIG. 9 is a side elevational view of the second embodiment of the translaminar screw illustrated in FIG. 8.

FIG. 10 is a sectional elevational view of the second embodiment of the translaminar screw illustrated in FIGS. 8 and 9.

FIG. 11 is a perspective view of a third embodiment of a translaminar screw in accordance with this invention.

FIG. 12 is a side elevational view of the third embodiment of the translaminar screw illustrated in FIG. 11.

FIG. 13 is a sectional elevational view of the third embodiment of the translaminar screw illustrated in FIGS. 11 and 12.

FIG. 14 is a perspective view of a fourth embodiment of a translaminar screw in accordance with this invention.

FIG. 15 is a side elevational view of the fourth embodiment of the translaminar screw illustrated in FIG. 14.

FIG. 16 is a sectional elevational view of the fourth embodiment of the translaminar screw illustrated in FIGS. 14 and 15.

FIG. 17 is a perspective view of a first embodiment of a working cannula in accordance with this invention.

FIG. 18 is a sectional elevational view of the first embodiment of the working cannula illustrated in FIG. 17.

FIG. 19 is a perspective view of a first embodiment of a trocar in accordance with this invention.

FIG. 20 is a side elevational view of the first embodiment of the trocar illustrated in FIG. 19.

FIG. 21 is a perspective view of an assembly of the working cannula illustrated in FIGS. 17 and 18 and the trocar illustrated in FIGS. 19 and 20.

FIG. 22 is a side elevational view of the assembly of the working cannula and trocar assembly illustrated in FIG. 21.

FIG. 23 is a perspective view of an insertion trocar in accordance with this invention.

FIG. 24 is a sectional elevational view of the insertion trocar illustrated in FIG. 23.

FIG. 25 is an enlarged perspective view of an end of the insertion trocar illustrated in FIGS. 23 and 24.

FIG. 26 is a perspective view of an assembly of the working cannula illustrated in FIGS. 17 and 18 and the insertion trocar illustrated in FIGS. 23, 24, and 25.

FIG. 27 is a sectional elevational view of the trocar assembly illustrated in FIG. 26.

FIG. 28 is a perspective view of an assembly of the trocar assembly illustrated in FIGS. 26 and 27 including a Kirschner wire.

FIG. 29 is an enlarged perspective view of an end of the trocar assembly and the Kirschner wire illustrated in FIG. 28.

FIG. 30 is a perspective view of a translaminar screw driver assembly in accordance with this invention.

FIG. 31 is a perspective view of a screw driver shaft for the translaminar screw driver assembly illustrated in FIG. 30.

FIG. 32 is an enlarged perspective view of an end of the screw driver shaft illustrated in FIG. 31.

FIG. 33 is a side elevational view of a drill bit in accordance with this invention.

FIGS. 34 through 40 illustrate a method for performing a minimal invasive surgical technique for placement of a translaminar screw on a spine in accordance with this invention.

FIG. 41 illustrates how translaminar screws in accordance with this invention can be used in the cervical spine to facilitate fusion of C1 and C2 vertebrae.

FIGS. 42 and 43 illustrate how translaminar screws in accordance with this invention can be used for fixation of the odontoid peg fracture, which is a fracture of the C2 vertebra.

FIGS. 44 and 45 illustrate how translaminar screws in accordance with this invention can be used for fixation of a sacroiliac joint.

FIG. 46 shows an alignment sensor attached to a biopsy needle or a cannula that allows a surgeon to achieve the correct anatomical trajectory based on a pre-operative planning study.

### DETAILED DESCRIPTION OF THE PREFERRED EMBODIMENTS

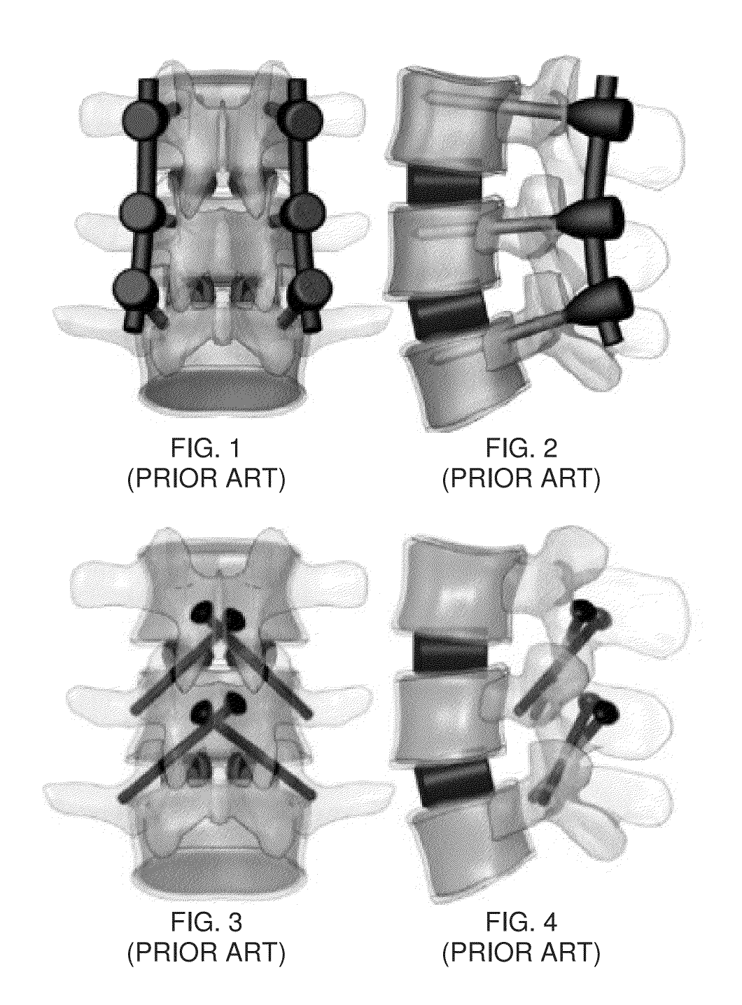

FIGS. 1 and 2 illustrate a conventional pedicle screw stabilization structure that is formed from a stainless steel or titanium alloy material, along with a portion of a spine showing a pedicle screw stabilization structure. FIGS. 3 and 4 illustrate a portion of a spine showing a conventional translaminar screw fixation technique developed by Fritz Magerl.

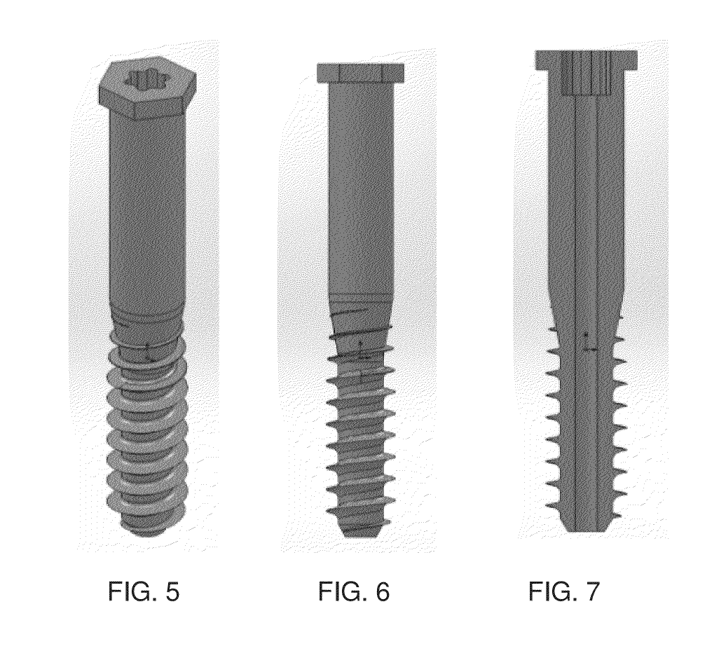

FIGS. 5, 6, and 7 illustrate a first embodiment of a cannulated translaminar screw, indicated generally at 10, in accordance with this invention. In this first embodiment of the invention, the screw 10 includes a head portion 11, a non-threaded portion 12, and a threaded portion 13. As best shown in FIG. 5, the head portion 11 of the screw 10 has an outer surface and an inner driving structure. In the illustrated embodiment, the outer surface of the head portion 11 is generally hexagonal in shape and the inner driving structure is generally star-shaped, although any other shapes may be provided. In the illustrated embodiment, the size of the head portion 11 of the screw 10 is somewhat larger than the size of the non-threaded portion 12. Thus, the outer surface of the screw 10 is stepped from the head portion 11 to the non-threaded portion 12. Also, in the illustrated embodiment, the diameter of the non-threaded portion 12 is somewhat larger than the diameter of the threaded portion 13. Thus, the outer surface of the screw 10 is tapered from the non-threaded portion 12 to the threaded portion 13. The thread provided on the threaded portion 13 of the screw 10 can having any desired shape or configuration including, for example, a single lead, a double lead, or a quad lead. A passageway 14 is formed through the screw 10 from the head portion 11 through the non-threaded portion 12 to the threaded portion 13 for a purpose that will be explained below. The entire screw 10 is formed from a polymer material such as, for example, PEEK, PLLA, PCL, carbon fiber PEEK, and the like, and can be used as a translaminar screw in the field of surgical spine treatment and for other applications.

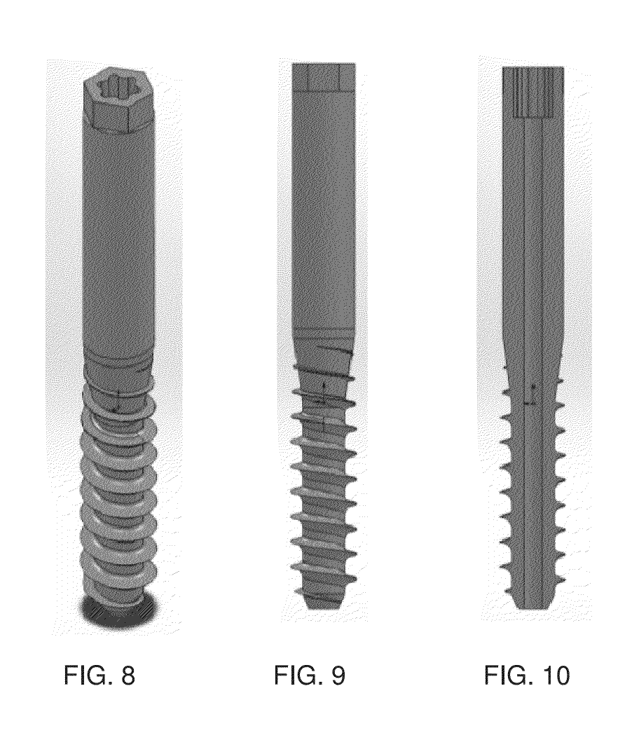

FIGS. 8, 9, and 10 illustrate a second embodiment of a cannulated translaminar screw, indicated generally at 20, in accordance with this invention. In this second embodiment of the invention, the screw 20 includes a head portion 21, a non-threaded portion 22, and a threaded portion 23. As best shown in FIG. 8, the head portion 21 of the screw 20 has an outer surface and an inner driving structure. In the illustrated embodiment, the outer surface of the head portion 21 is generally hexagonal in shape and the inner driving structure is generally star-shaped, although any other shapes may be provided. In the illustrated embodiment, the size of the head portion 21 of the screw 20 is approximately the same size as the size of the non-threaded portion 22. Thus, the outer surface of the screw 20 is essentially flush with the head portion 21 to the non-threaded portion 22. Also, in the illustrated embodiment, the diameter of the non-threaded portion 22 is somewhat larger than the diameter of the threaded portion 23. Thus, the outer surface of the screw 20 is tapered from the non-threaded portion 22 to the threaded portion 23. The thread provided on the threaded portion 23 of the screw 20 can having any desired shape or configuration including, for example, a single lead, a double lead, or a quad lead. A passageway 24 is formed through the screw 20 from the head portion 21 through the non-threaded portion 22 to the threaded portion 23 for a purpose that will be explained below. The entire screw 20 is formed from a polymer material such as, for example, PEEK, PLLA, PCL, carbon fiber PEEK, and the like, and can be used as a translaminar screw in the field of surgical spine treatment and for other applications.

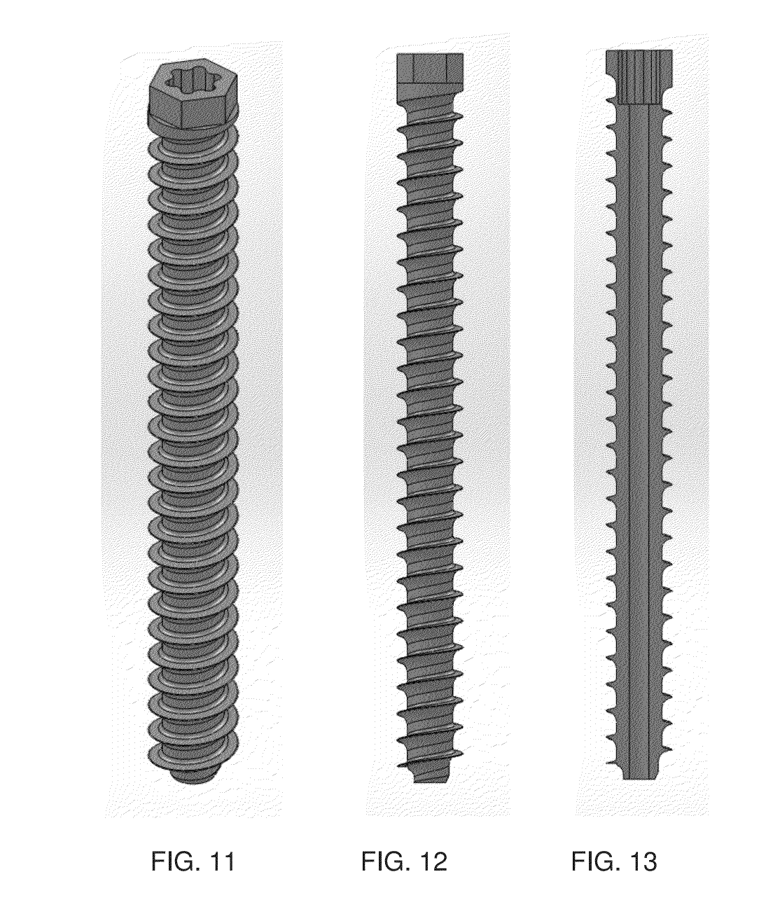

FIGS. 11, 12, and 13 illustrate a third embodiment of a cannulated translaminar screw, indicated generally at 30, in accordance with this invention. In this third embodiment of the invention, the screw 30 includes a head portion 31 and a threaded portion 33. As best shown in FIG. 11, the head portion 31 of the screw 30 has an outer surface and an inner driving structure. In the illustrated embodiment, the outer surface of the head portion 31 is generally hexagonal in shape and the inner driving structure is generally star-shaped, although any other shapes may be provided. In the illustrated embodiment, the size of the head portion 31 of the screw 30 is approximately the same size as the size of the non-threaded portion 32. Thus, the outer surface of the screw 30 is essentially flush with the head portion 31 to the non-threaded portion 32. Also, in the illustrated embodiment, the thread provided on the threaded portion 33 of the screw 30 can having any desired shape or configuration including, for example, a single lead, a double lead, or a quad lead. A passageway 34 is formed through the screw 20 from the head portion 31 to the threaded portion 33 for a purpose that will be explained below. The entire screw 30 is formed from a polymer material such as, for example, PEEK, PLLA, PCL, carbon fiber PEEK, and the like, and can be used as a translaminar screw in the field of surgical spine treatment and for other applications.

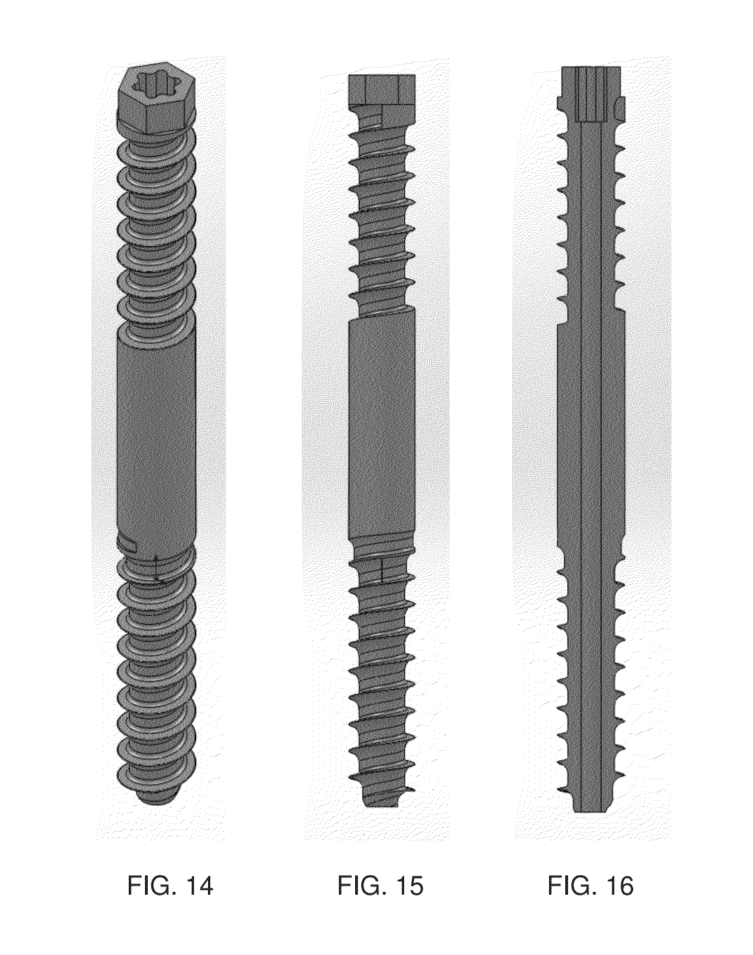

FIGS. 14, 15, and 16 illustrate a fourth embodiment of a cannulated translaminar screw, indicated generally at 40, in accordance with this invention. In this fourth embodiment of the invention, the screw 40 includes a head portion 41, a non-threaded portion 42, and a threaded portion 43. As best shown in FIG. 14, the head portion 41 of the screw 40 has an outer surface and an inner driving structure. In the illustrated embodiment, the outer surface of the head portion 41 is generally hexagonal in shape and the inner driving structure is generally star-shaped, although any other shapes may be provided. In the illustrated embodiment, the size of the head portion 41 of the screw 40 is approximately the same size as the size of the non-threaded portion 42. Thus, the outer surface of the screw 40 is essentially flush with the head portion 41 to the non-threaded portion 42. Also, in the illustrated embodiment, the non-threaded portion 42 is provided within an intermediate region of the threaded portion 43. The thread provided on the threaded portion 43 of the screw 40 can having any desired shape or configuration including, for example, a single lead, a double lead, or a quad lead. A passageway 44 is formed through the screw 40 from the head portion 41 through the non-threaded portion 42 to the threaded portion 43 for a purpose that will be explained below. The entire screw 40 is formed from a polymer material such as, for example, PEEK, PLLA, PCL, carbon fiber PEEK, and the like, and can be used as a translaminar screw in the field of surgical spine treatment and for other applications.

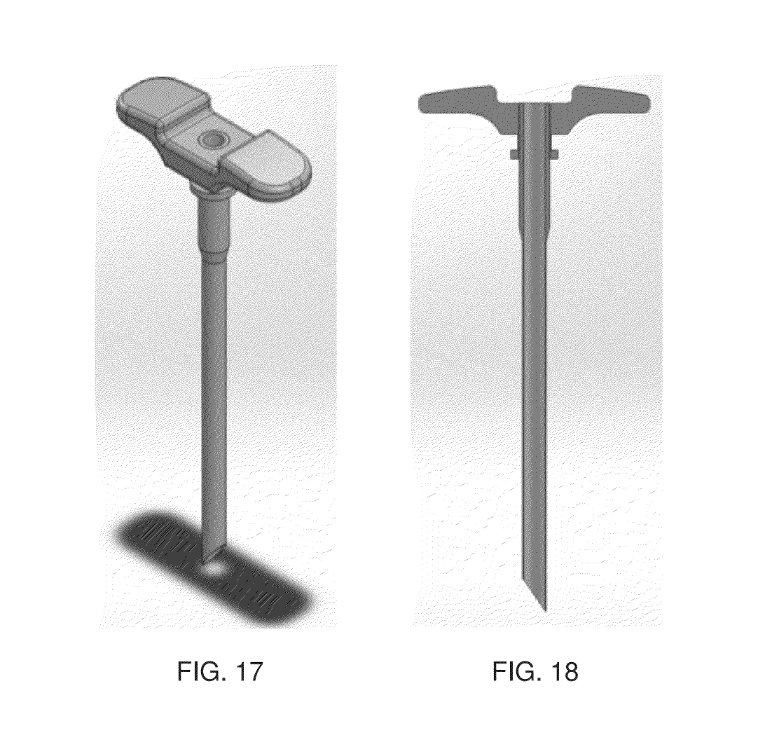

FIGS. 17 and 18 illustrate a first embodiment of a working cannula, indicated generally at 50, in accordance with this invention. In the illustrated embodiment, the working cannula 50 includes a handle portion 51, a cannula portion 52, and a sharp tip 53. As best shown in FIG. 18, the cannula portion 52 is tapered from the handle portion 51 to the tip portion 53, although such is not required. A passageway 54 is formed through the working cannula 50 from the handle portion 51 through the cannula portion 52 to the sharp tip 53 for a purpose that will be explained below. The cannula portion 52 of the working cannula 50 can vary in length from about 100 mm to about 200 mm and is preferably about 120 mm. The overall length of the working cannula 50 can also vary, but is preferably about 150 mm. The cannula portion 52 defines an inner diameter that can vary with the size of the translaminar screw used therewith, as will be described below. For example, the inner diameter of the cannula portion 52 can be about 7 mm ID when a translaminar screw or other implant of about 4.5 mm is used.

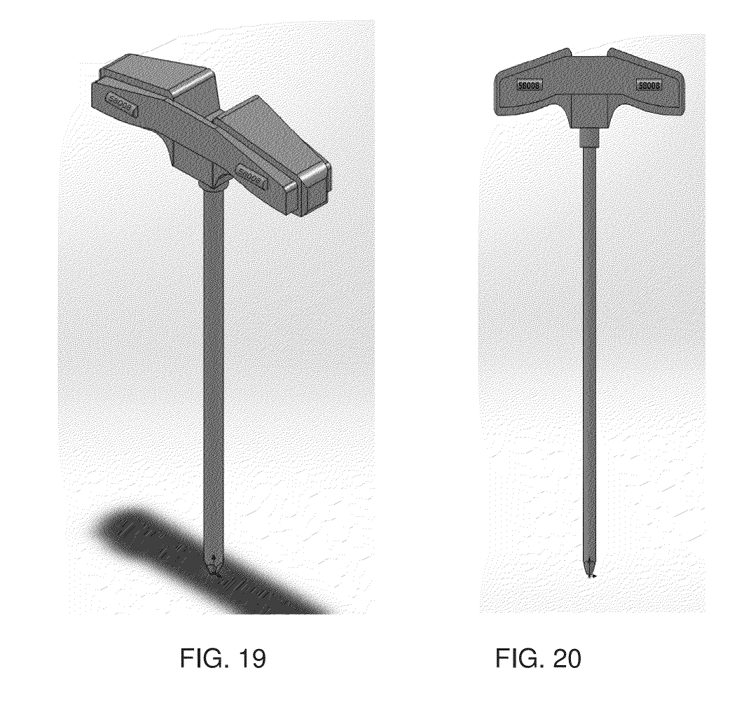

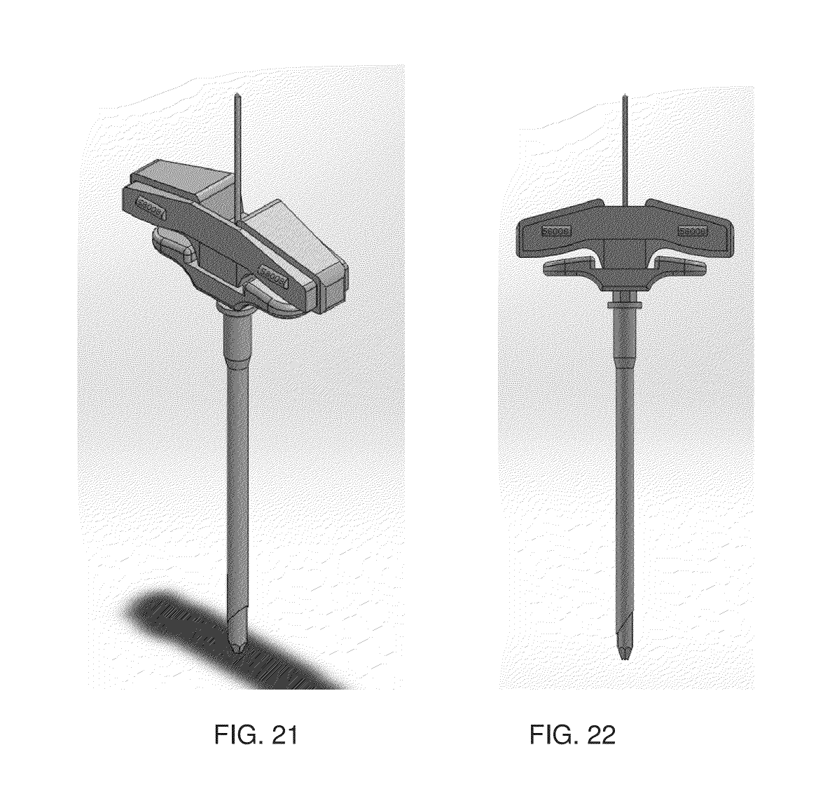

FIGS. 19 and 20 illustrate a first embodiment of a trocar, indicated generally at 60, in accordance with this invention. In the illustrated embodiment, the trocar 60 includes a handle portion 61 and a shaft portion 62 that terminates in a sharp tip 63. A passageway (not shown) is formed through the shaft portion 62 from the handle portion 61 to the sharp tip 63 to accommodate the passage of a conventional Kirschner wire 64 (see FIGS. 21 and 22) therethrough. FIGS. 21 and 22 illustrate the assembly of the working cannula 50 illustrated in FIGS. 17 and 18 and the trocar 60 illustrated in FIGS. 19 and 20. As shown therein, the shaft portion 62 of the trocar 60 can be inserted through the cannula portion 52 of the working cannula 50 such that the sharp tip 63 of the trocar 60 extends from the sharp tip 53 of the working cannula 50. The Kirschner wire 64 is shown in use with the assembly of the working cannula 50 and the trocar 60.

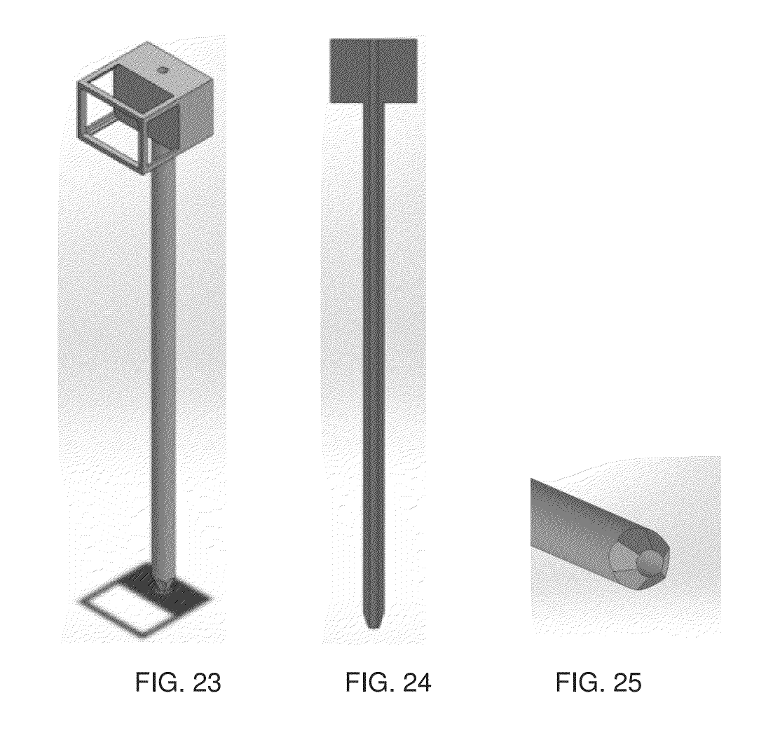

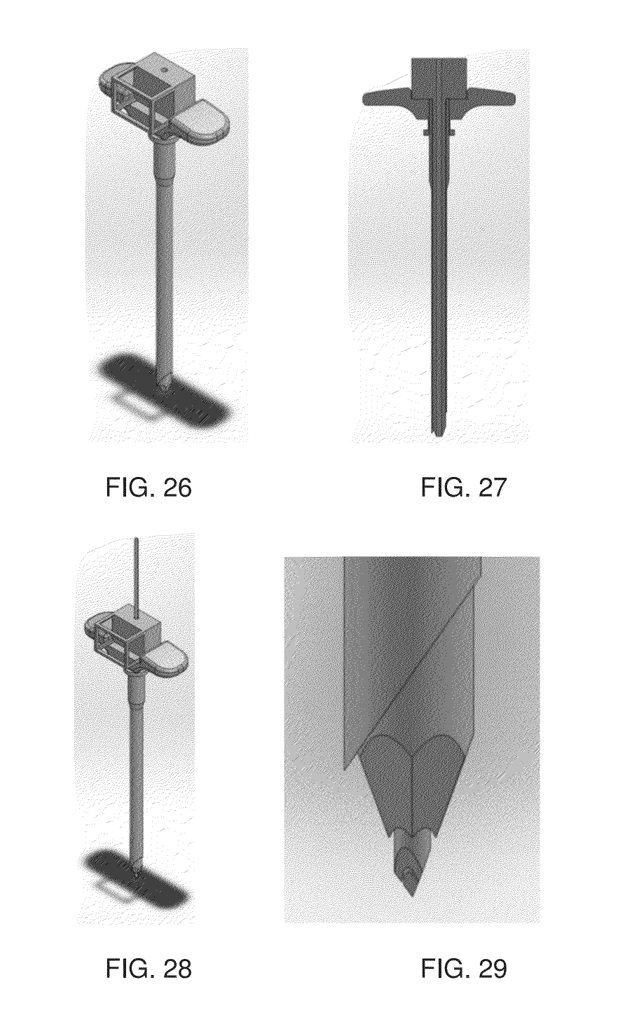

FIGS. 23, 24, and 25 illustrate a second embodiment of a trocar, indicated generally at 70, in accordance with this invention. In the illustrated embodiment, the trocar 70 includes a caged handle portion 71 and a shaft portion 72 that terminates in a sharp tip 73. A passageway 74 is formed through the shaft portion 72 from the caged handle portion 71 to the sharp tip 73 to accommodate the passage of a conventional Kirschner wire 74 (see FIGS. 28 and 29) therethrough. FIGS. 26 through 29 illustrate the assembly of the working cannula illustrated in FIGS. 17 and 18 and the trocar 70 illustrated in FIGS. 23, 24, and 25. As shown therein, the shaft portion 72 of the trocar 70 can be inserted through the cannula portion 52 of the working cannula 50 such that the sharp tip 73 of the trocar 70 extends from the sharp tip 53 of the working cannula 50. The Kirschner wire 74 is shown in use with the assembly of the working cannula 50 and the trocar 70. The caged handle portion 71 of the trocar 70 is provided to facilitate the attachment of an inclinometer (see 95 in FIG. 46) thereto for use during a surgical procedure. As will be explained further below, the inclinometer 95 is, of itself, conventional in the art and is adapted to generate an indication of the slope, tilt, angle, elevation, or depression of the trocar 70 relative to a reference line defined (in this instance) by gravity. Thus, the assembly of the working cannula 50 and the trocar 60 or 70 can be properly and accurately positioned for and during use. Teeth provided at the tip of the trocar 70 (best shown in FIG. 25) allow for proper grip into the bone. This part can be used as a blunt dissection tool and to prevent tissue from entering into the working cannula 50 during use.

In use, the trocar 70 slides into the working cannula 50 and can be locked into place to prevent sliding and rotation during surgery. One or more Kirschner wires can then be inserted into the trocar 70. For example, a relatively thick Kirschner wire can provides stability to a relatively thin Kirschner wire when it is inserted into the bone. The Kirschner wires may have diamond tipped ends (see FIG. 29) to provide proper grip and accurate placement into the bone.

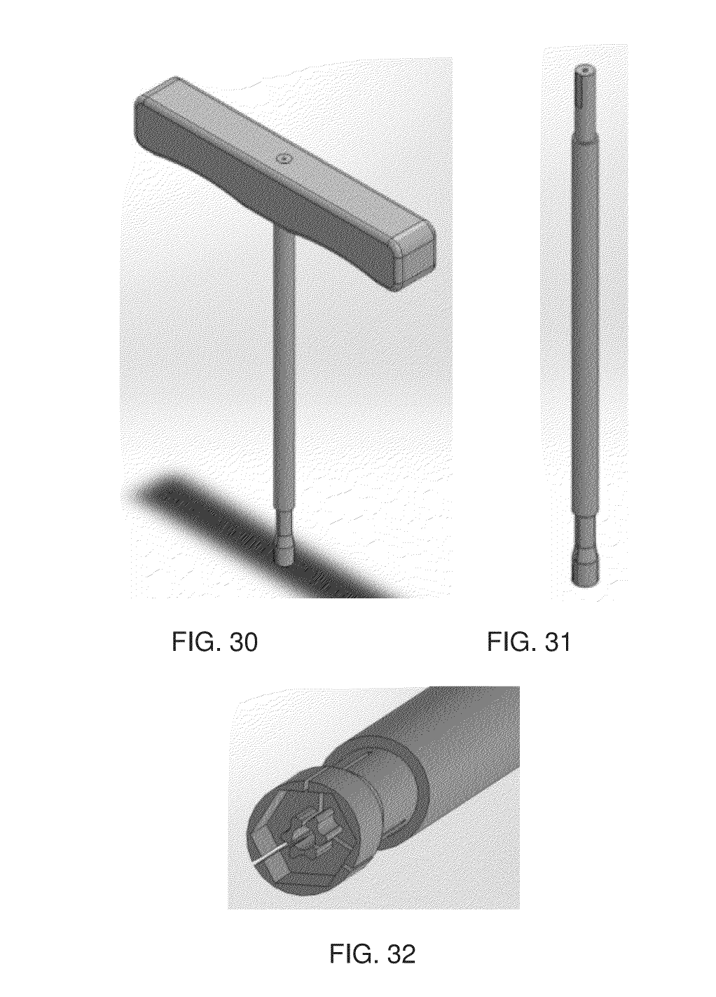

FIG. 30 illustrates a translaminar screwdriver assembly, indicated generally at 80, in accordance with this invention. In the illustrated embodiment, the screwdriver assembly 80 includes a handle portion 81 and a shaft portion 82 that terminates in a driver tip 83. The handle portion 81 is preferably relative large to facilitate grasping and applying rotational force by a user. The shaft portion 82 of the screwdriver assembly 80 can be of any desired length. The driver tip 83 of the screwdriver assembly 80 is shaped to be complementary to the inner driving structures of the head portions of the translaminar screws described above. As a result, a translaminar screw can be inserted through the cannula portion 52 of the working cannula 50 and rotatably driven into the bone by the screwdriver assembly 80. If desired, the driver tip 83 of the screwdriver assembly 80 may be provided with one of more splits 83a (three are shown in the illustrated embodiment) that allow the driver tip 83 to frictionally engage the outer surface of the head portion of the translaminar screw being driven into the bone. A passageway 84 may be formed through the screwdriver assembly 80 from the handle portion 81 through the shaft portion 82 to the driver tip 83 to accommodate a Kirschner wire (not shown) for facilitating alignment.

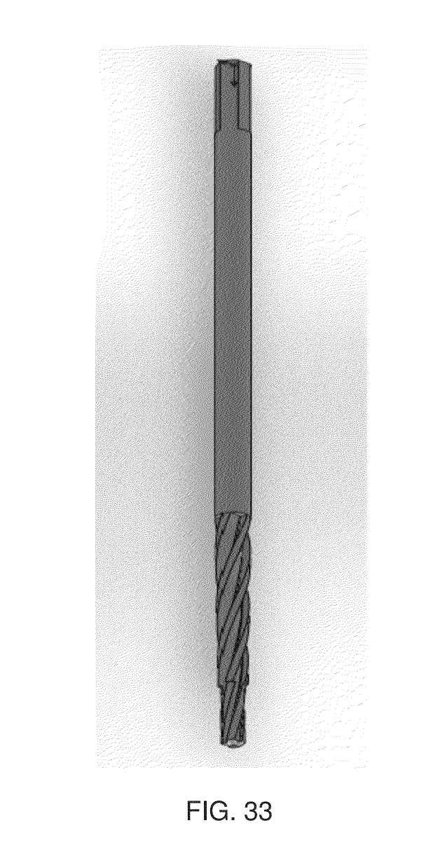

FIG. 33 is a side elevational view of a drill bit, indicated generally at 90, that can be used for surgery in accordance with this invention. In the illustrated embodiment, the drill bit 90 includes an engagement portion 91, a shaft portion 92, a relatively large diameter drill portion 93, and a relatively small diameter portion 94. The engagement portion 91 is provided to facilitate the connection of the drill 90 with a source of rotational power (not shown). The relatively large diameter drill portion 93 is provided to create a cavity for larger diameter portion of the translaminar screw (such as the non-threaded portion 12 of the translaminar screw 10 illustrated in FIGS. 5, 6, and 7), while the relatively small diameter portion 94 is provided to create a cavity for smaller diameter portion of the translaminar screw (such as the threaded portion 13 of the translaminar screw 10 illustrated in FIGS. 5, 6, and 7).

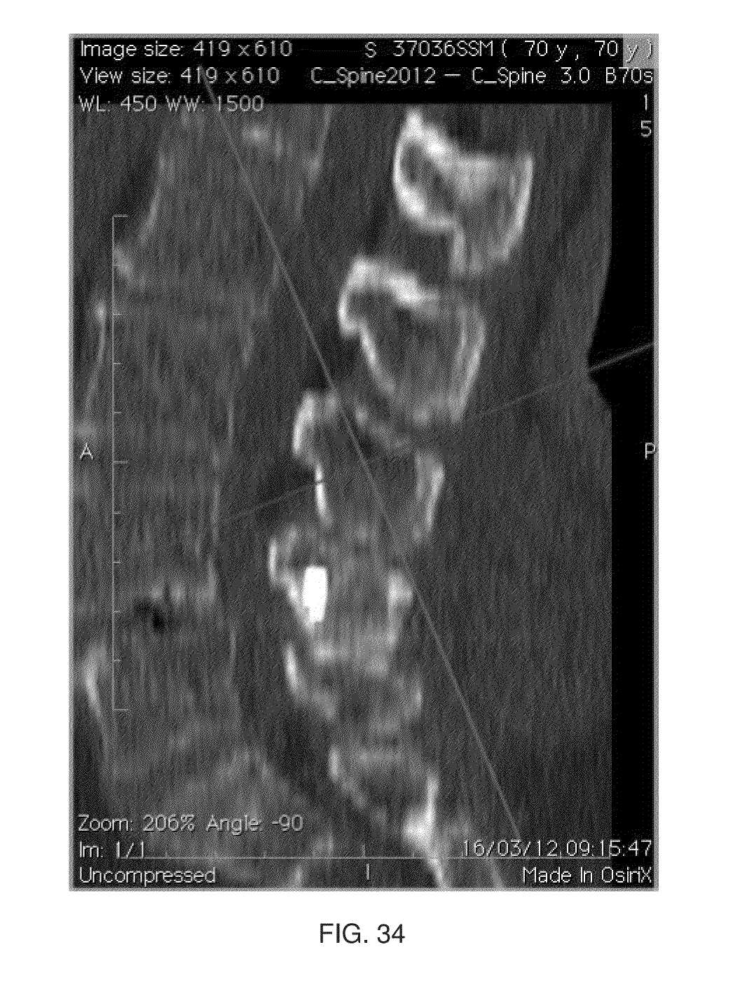

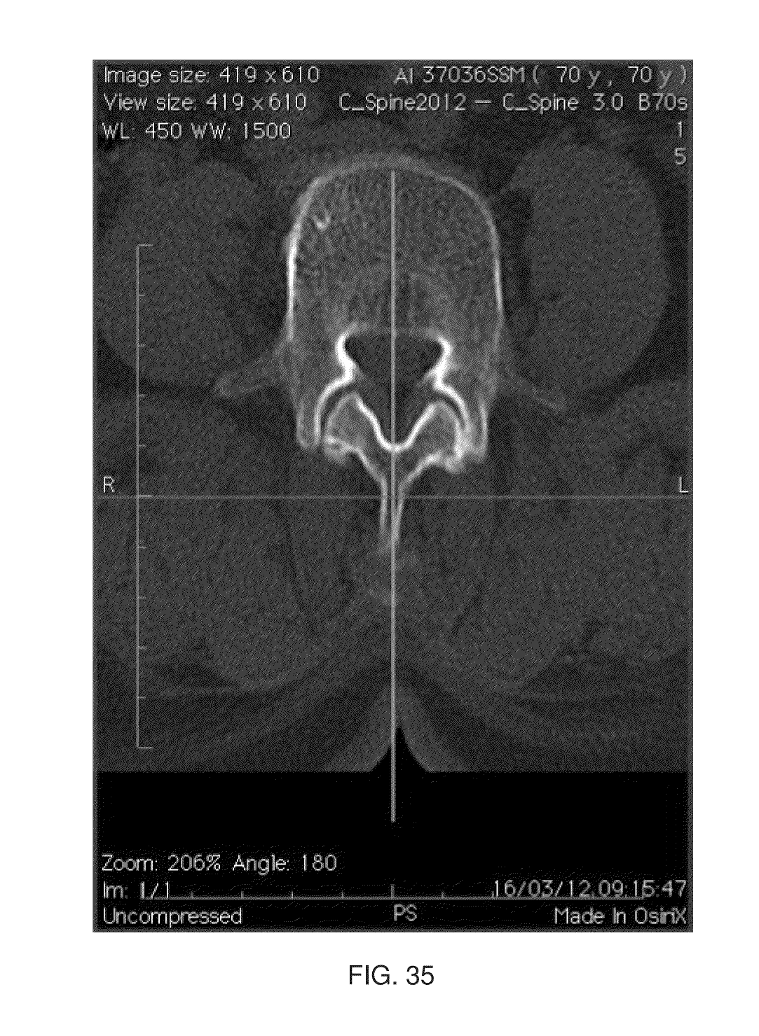

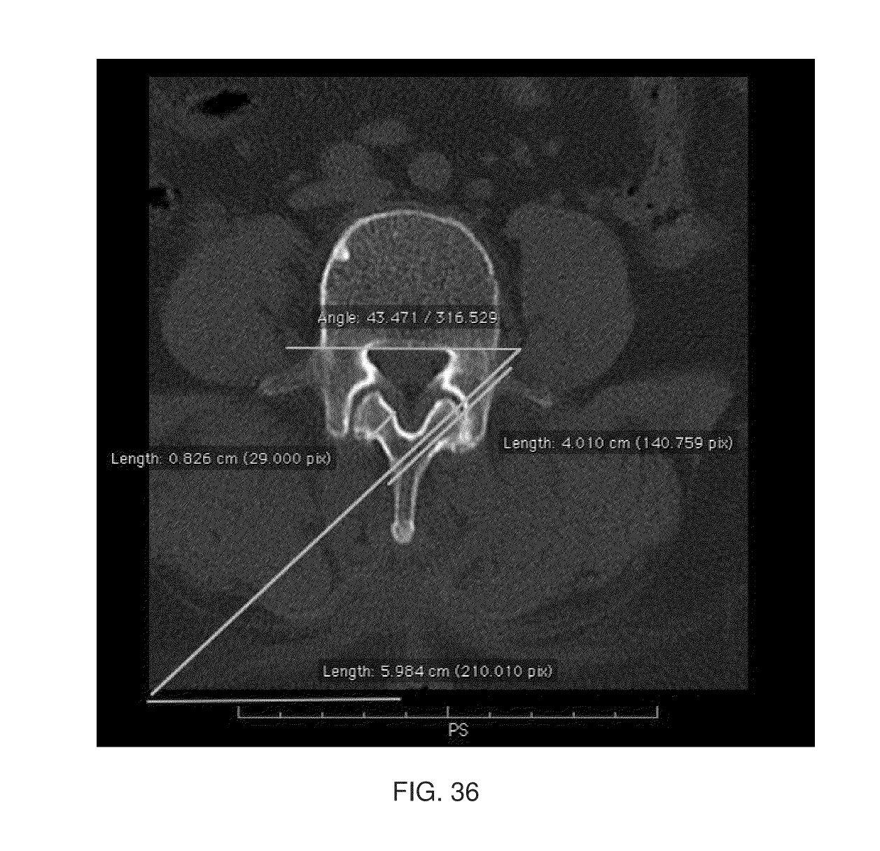

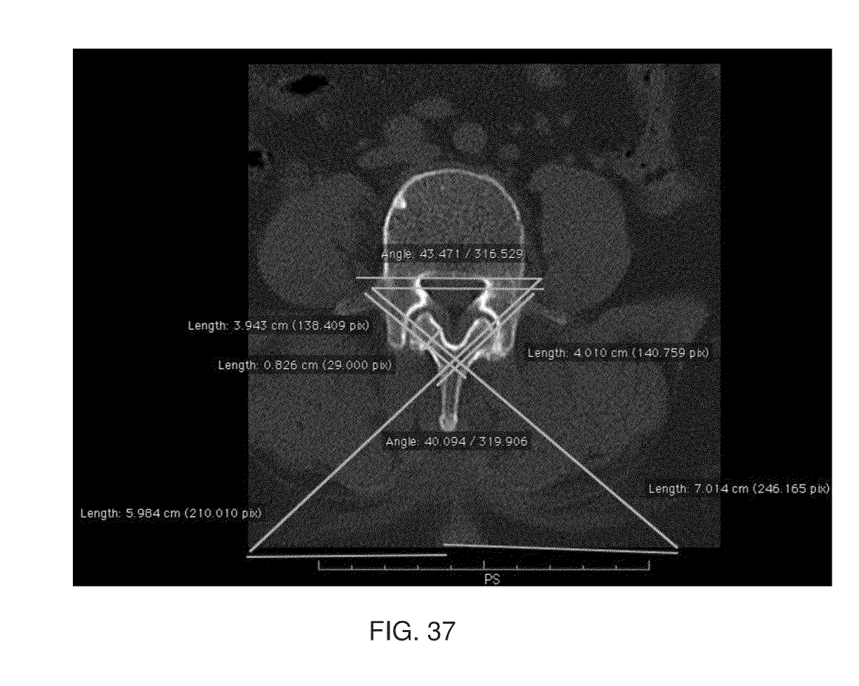

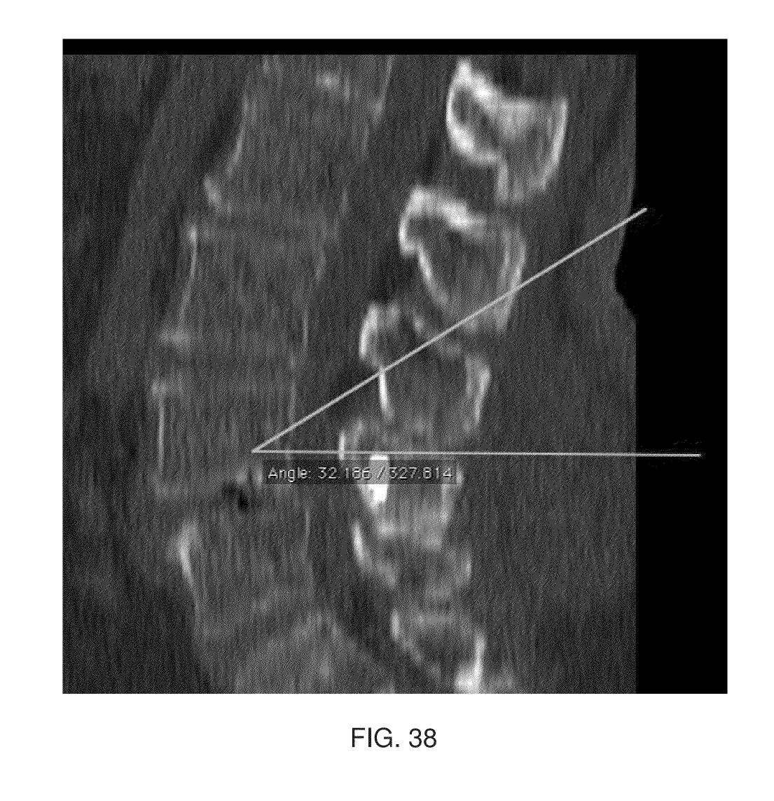

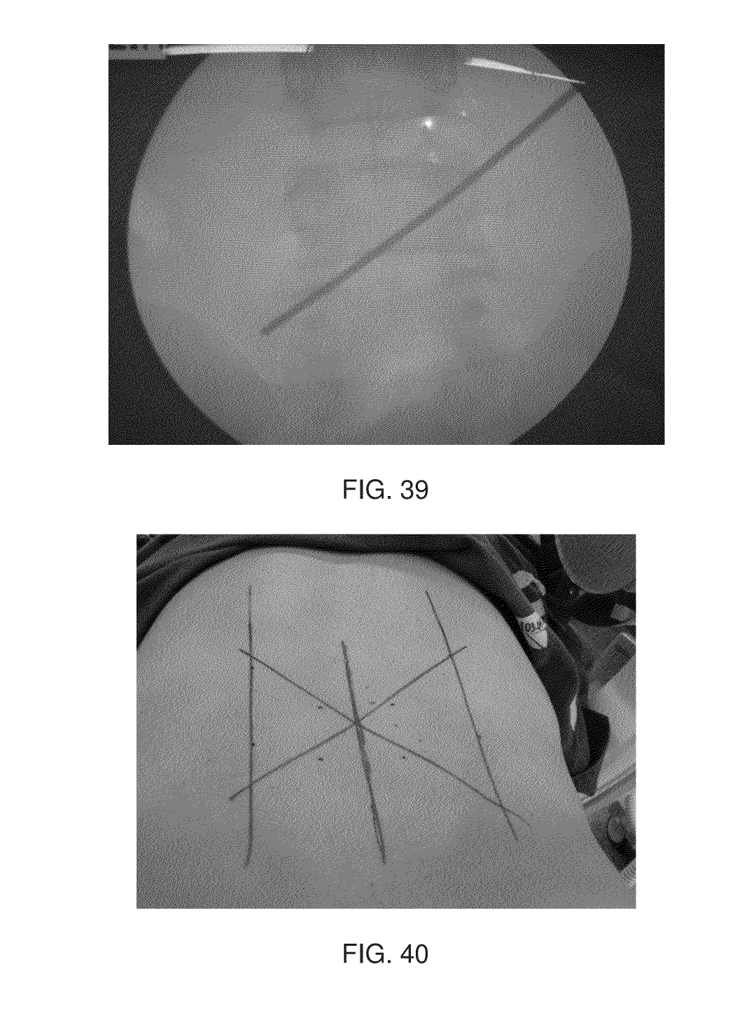

FIGS. 33 through 40 illustrate a method for performing a minimal invasive surgical technique for placement of a translaminar screw in accordance with this invention. Surgical preplanning can be done using a conventional CT scan using simple and conventional software. As shown in these drawings, the desired angulation of the translaminar screw can be calculated. Thereafter, the inclination angle can be calculated. These calculations allows the surgery to be planned with minimal opportunity for error during the minimal invasive spine surgery placement of the translaminar screw. This angulation preplanning is then transferred as marking on the skin as shown in FIG. 40.

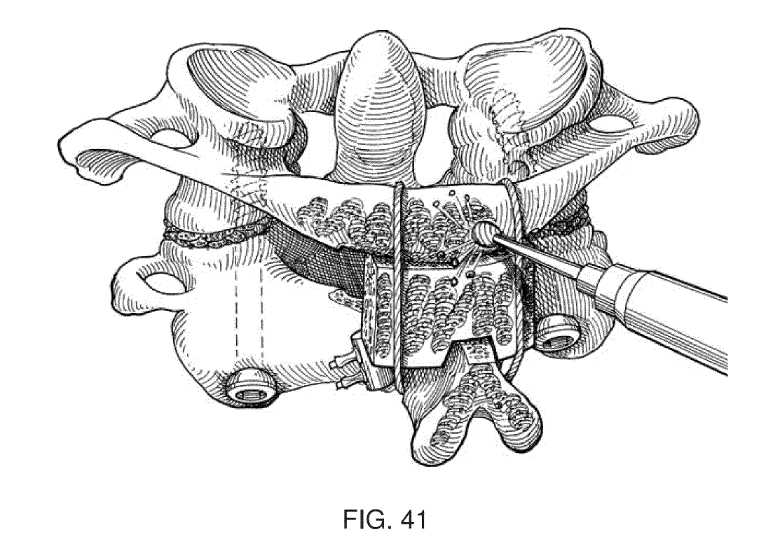

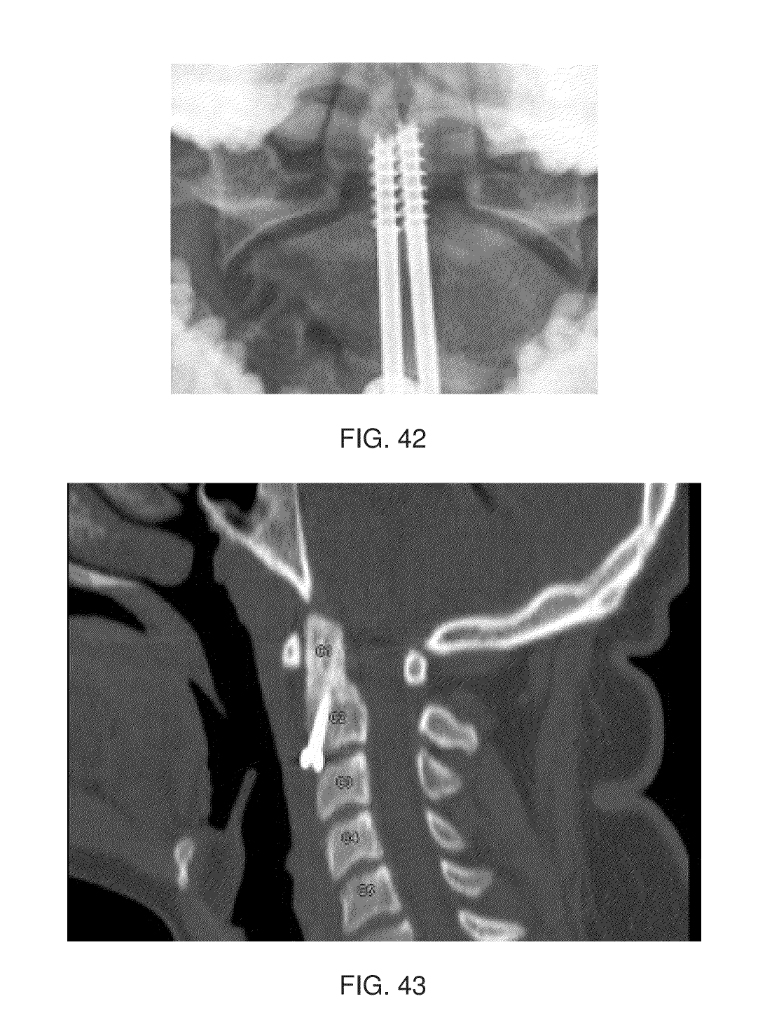

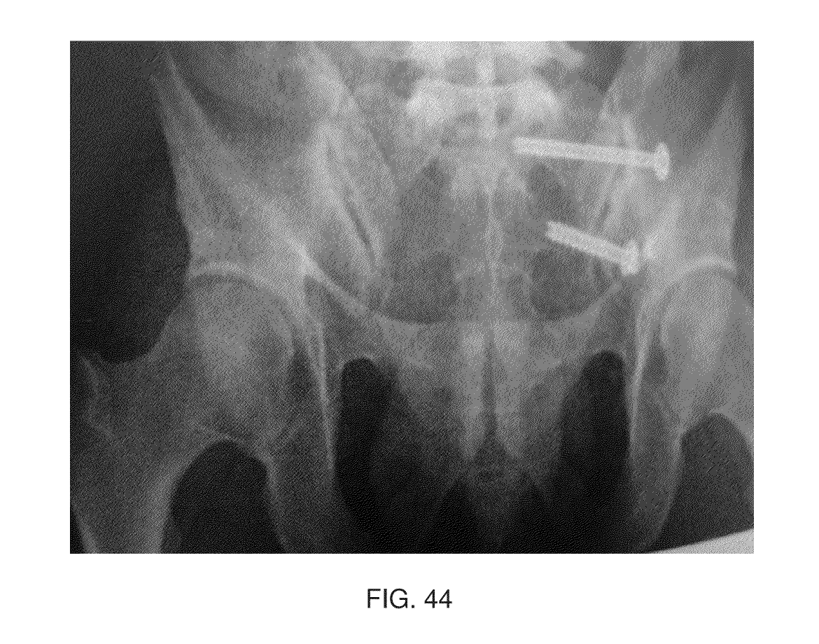

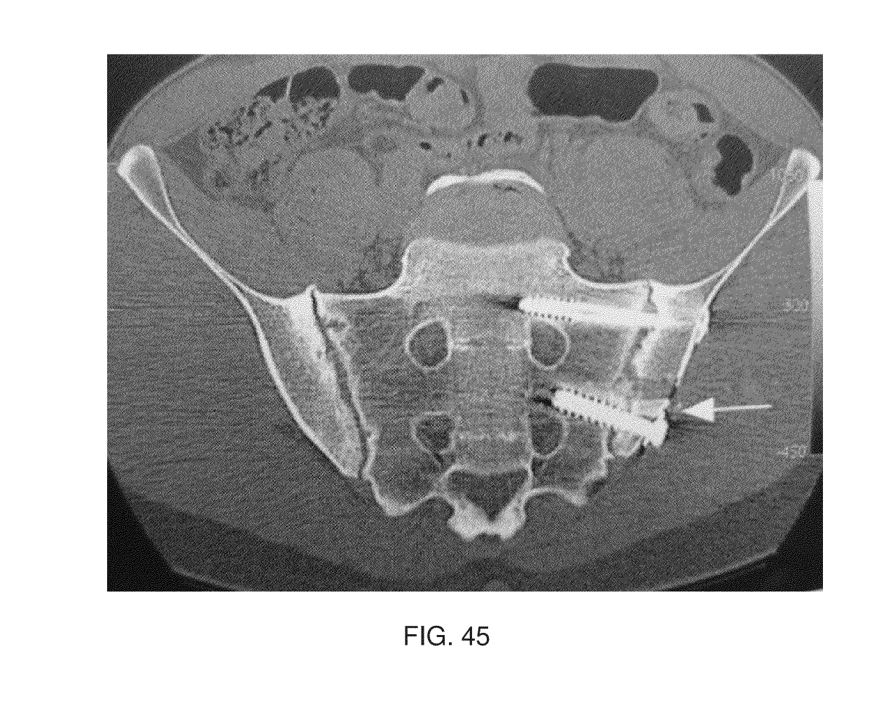

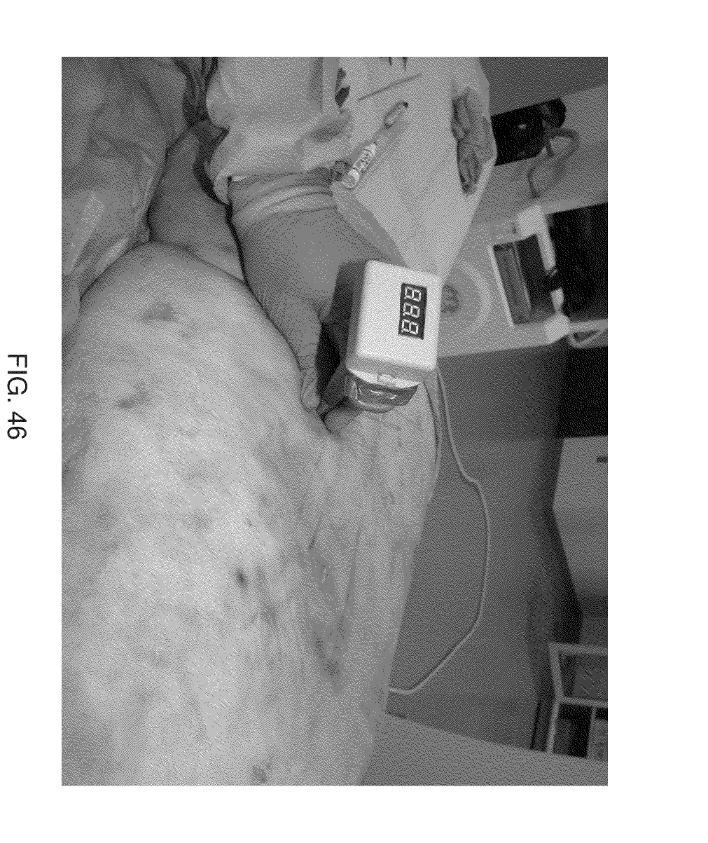

Many other applications of this polymer osteosynthesis screw (formed from PEEK, PEAK, or carbon fiber) are within the scope of this invention. For example, as shown in FIG. 41, these screws can be used in the cervical spine to do fusion of C1 and C2 vertebrae. FIGS. 42 and 43 show how these screws can be used for fixation of the odontoid peg fracture with is fracture of the C2 vertebra. FIGS. 44 and 45 show how these screws can be used for fixation of a sacroiliac joint. The screw can also be used for many other orthopedic application, such as wrist joint stability and ankle joint stability, as it would allow the joint to be stabilized while, at the same time, allowing function movement, thereby preventing fusion from occurring. The screw can also be used for osteoporotic fixation of various orthopedic fractures and surgical procedure with low quality bone.

Additionally, the screws of this invention may be used in transforaminal lumbar interbody fusion (TLIF) surgeries, anterior lumbar interbody fusion (ALIF) surgeries, extreme lateral interbody fusion (XLIF) surgeries, and other surgical procedures. Similarly, the screws of this invention may also be used in nucleus replacement, total disc replacement, and annular repair surgical procedures.

FIG. 46 shows an alignment sensor, such as a conventional inclinometer 95, that is attached the caged handle portion 71 of the trocar 70 for use during a surgical procedure. The inclinometer 95 is, as mentioned above, conventional in the art and is adapted to generate an indication of the slope, tilt, angle, elevation, or depression of the trocar 70 relative to a reference with respect to gravity. Thus, the assembly of the working cannula 50 and the trocar 60 or 70 can be properly and accurately positioned for and during use. The inclinometer 95 can alternatively be attached to a biopsy needle or other device that allows a surgeon to achieve the correct anatomical trajectory based on a preoperative planning study. It is very helpful during spine and orthopedic surgeries, mainly the minimally invasive and percutaneous ones, to avoid misplaced implants and the associated consequences. For example, it can be indicated to guide the placement of pedicle screws, transfacet screws, and translaminar facet screws in procedures that are commonly performed around the world. The goal of the illustrated alignment sensor is to achieve the lateral angle of a fluoroscopy guided surgery with accuracy.

Two other parameters that are important for a percutaneous placement (the caudal angle and the distance away from the midline) are also achieved during the preoperative planning study and drawn at the patient skin. One advantage of this new device is to allow a free hand navigation surgery without the necessity of a new skin incision to place the other techniques hardware and the easy way to handle it. The new device can be attached to all biopsy needle designs available on the market or adapted to customized ones or cannulas. The new alignment sensor is a very simple technological solution based on electronic components currently available, thus reducing its cost of manufacturing. The only simple orientation for the surgeon is to keep the patient position parallel to the operating surgery floor, avoiding an incorrect angle trajectory. Another big advantage of this device is to reduce the increased radiation exposure time for surgeons and patients, during minimally invasive and percutaneous surgery.

The illustrated alignment sensor 95 is an inclinometer to use for in a surgical application. The inclinometer is capable of measure an angle between +90° and −90° from the referential ground plane (lateral angle). The measured angle assists the surgeon to introduce and position a needle during a surgical procedure that demands precise lateral angle positioning. Despite the rotation on its perpendicular axis, the inclinometer will continually show the lateral angle referred to ground plane. The inclinometer can remain off while not in use and will turn on its display when tapped consistently in its radial direction, like hitting a coin in a table. Once turned on it will remain in this state, showing the measured angle on display, while it has internal power to do so and while the absolute measured angle is greater than 10°. If the inclinometer is positioned below absolute 10° for fifteen seconds or other predetermined period of time, it will turn off and wait for another initialization with a radial tap.

Although it can be applied to other type of surgeries, its first application is in spine soft fusion procedures, when the cannula that orients all the procedure is precisely positioned. In order to the readings be accurate with the necessary angle, the floor of the surgical room must be parallel to the earth's ground plane and the patient's back must be positioned in parallel with earth's ground plane as well.

The inclinometer 95 can have an elliptical coin form factor that accommodates an easily readable luminous display with 2.1 digits for positive angles or −1.1 digits for negative angles above −10°, or −2 digits for negative angles below −10°. It can also have an interchangeable clip that adjusts and grips in the cannula in order to keep the inclinometer in an orthogonal angle in reference to the cannula extension (then the measured angle corresponds to the lateral angle of the cannula itself in reference to the earth's ground plane). It could have distinct body colors and formats.

The sensing element can be a 3-axis micro electromechanical system accelerometer, which is capable to sense the vector of gravity. Desired features for this implementation include:
3-axis;
small size;
low full scale (between 2 g and 5 g) with high sensitivity;
low noise;
low power consumption;
wide range of work voltage, to operate directly from the battery;
good long term stability.

The processing is made by a microcontroller unit (MCU) which runs a firmware that gives all the described functionality from the data collected from the sensing element. This MCU gives flexibility and adaptability to the design and thanks to a arithmetic unit capable of run the vectorial math and a set of integrated peripherals it allows the design of a very compact design. The desired MCU features in this design can include:
small size;
low power consumption when processing;
extremely low power consumption while standing-by;
32 KB of internal non-volatile memory (FLASH) for program and configuration;
8 KB of internal data SRAM;
capability to run emulated floating-point operations with power and time efficiency, which a low-power 32-bit 12 Mhz MCU core with one-cycle multiplier should be able to do;
direct interface to selected MEMS accelerometer;
battery monitoring interface;
display drive capability;
wake-up from power-down mode through an interrupt pin;
timer;
no pin conflicts.

The main floating-point operation can be defined by the equation:

$$θ = {tan}^{- 1} ( \frac{{A}_{X , OUT}}{\sqrt{{A}_{Y , OUT}^{2} + {A}_{Z , OUT}^{2}}} )$$

where:
θ=measured angle
tan⁻¹=arctangent operation
A_{X,OUT}=value read in X-axis
A_{Y,OUT}=value read in Y-axis
A_{Z,OUT}=value read in Z-axis

The principle and mode of operation of this invention have been explained and illustrated in its preferred embodiments. However, it must be understood that this invention may be practiced otherwise than as specifically explained and illustrated without departing from its spirit or scope.

What is claimed is:

1. A screw that is used in the field of surgical spine treatment and is formed from a polymer material.

2. The screw defined in claim 1 wherein the screw is formed from one of a PEEK, PEAK, PCL, or carbon fiber material.

3. The screw defined in claim 1 wherein the screw has two mechanisms on a head to drive it into bone.

4. The screw defined in claim 1 wherein the screw has a conical inclination when it transitions from a threaded portion to a non-threaded portion.

5. The screw defined in claim 1 wherein the screw has a head portion that is the same size as a shaft portion.

6. The screw defined in claim 1 wherein the screw has a threaded portion and a non-threaded portion that is disposed within an intermediate region of the threaded portion.

7. The screw defined in claim 1 wherein the screw is used for translaminar fusion of lumbar spine, lateral mass fusion of C1/C2 vertebrae, dynamic stabilization of sacroiliac joints and C2 peg fracture.

8. A screwdriver having a mechanism to drive the screw defined in claim 1 into bone.

9. The screwdriver defined in claim 8 including a mechanism to lock the screw onto the screwdriver.

10. An alignment sensor attached to a biopsy needle or a cannula that allows a surgeon to achieve the correct anatomical trajectory based on a pre-operative planning study.

11. The alignment sensor defined in claim 10 wherein the alignment sensor is an inclinometer.

12. The alignment sensor defined in claim 10 wherein the inclinometer is used in all surgical procedures to achieve alignment and correct positioning of minimal invasive working portals.

13. The alignment sensor defined in claim 10 wherein the inclinometer can sense alignment in X and Y axis.

14. The alignment sensor defined in claim 10 wherein the inclinometer is used to pass through the pedicle of a vertebrae to accurately place the working cannula or wire.

15. A method of performing a surgical procedure comprising the step of attaching a polymer screw that is formed from one of a PEEK, PEAK, PCL, or a carbon fiber material to a bone.
